(19) United States
(12) Patent Application Publication
(10) Pub. No.: US 2024/0264071 A1
(43) Pub. Date: Aug. 8, 2024

(54) IN-SITU DETECTION COMPONENTS FOR DISSOLVED GASES IN LIQUID-PHASE MEDIA AND PREPARATION METHODS THEREOF

(71) Applicant: CHONGQING UNIVERSITY, Chongqing (CN)

(72) Inventors: Fu WAN, Chongqing (CN); Weigen Chen, Chongqing (CN); Weiping Kong, Chongqing (CN); Pinyi Wang, Chongqing (CN); Lin Du, Chongqing (CN); Youyuan Wang, Chongqing (CN); Quan Zhou, Chongqing (CN)

(21) Appl. No.: 18/606,889

(22) Filed: Mar. 15, 2024

Related U.S. Application Data
(63) Continuation of application No. PCT/CN2023/100339, filed on Jun. 15, 2023.

(30) Foreign Application Priority Data
Jul. 8, 2022 (CN) 202210853431.7

(51) Int. Cl. G01N 21/01

(52) U.S. Cl. CPC G01N 21/01; G01N 2201/06113; G01N 2201/08

(57) ABSTRACT

An in-situ detection component for dissolved gases in liquid-phase media and preparation method of the in-situ detection component, the detection component is a drilled hollow-core light guiding device coated with a liquid-gas separation membrane on the outer surface. The liquid-gas separation membrane directly filters the gases dissolved in liquid-phase media into the drilled hollow-core light guiding device, where the laser interacts with the gases and generates the response signals that are transmitted through the hollow-core light guiding device, to realize the simultaneous dissolved gas separation and detection. The present application substantially reduces the time required for liquid-gas separation process in detection components, allowing for fast and accurate in-situ detection of dissolved gases, and integration of liquid-gas separation and detection procedures.

### CROSS-REFERENCE TO RELATED APPLICATIONS

This application is a bypass continuation application of PCT application no.: PCT/CN2023/100339. This application claims priorities from PCT Application PCT/CN2023/100339, filed Jun. 15, 2023, and from the Chinese patent application 202210853431.7 filed Jul. 8, 2022, the contents of which are incorporated herein in the entirety by reference.

### TECHNICAL FIELD

The present application relates to the technologies of detecting dissolved gases in liquid-phase media. Specifically, the present application relates to an in-situ detection component for dissolved gas in liquid medium and preparation methods of the component.

### BACKGROUND

For insulating oil of power transformers, dissolved gases include H2, CH4, C2H2, C2H4, C2H6, CO, CO2, etc. For electrolyte solutions for electrochemical energy storage systems, dissolved gases include C3H6, etc., while for drilling fluids, dissolved gases include hydrocarbon gases, and some landmark gases are in the deep sea, therefore reliable and high-precision detection technologies are the respective keys to realizing the early fault diagnosis of oil-immersed electrical equipment, safe operation of electrochemical energy storage systems, exploitation of petroleum and gas reserves and estimation of the production capacity, and exploration of submarine mineralized regions. However, conventional detection methods require separating gases from the liquid-phase media and then pumping the gases into gas chambers by the gas pumps for analysis by optical or electrochemical detection sensors. Such complex procedures result in poor accuracy, low reliability and real-time performance of dissolved gas detection, thus not able to reflect the real concentrations of dissolved gases.

Chinese Patent Application CN111579499A discloses a membrane component for the separation and detection of dissolved gases in transformer oil. However, in this solution, a hollow-core optical fiber is fixed and sealed in a sealing barrel with oil-gas separation membrane on both sides. The oil-gas separation membrane of this detection device is only applied to the two ends of the hollow-core optical fiber, resulting in small gaps between membrane and fiber. Gases must fill up the gaps before entering the hollow-core optical fiber for detection. This means that detection needs more transformer fault gases and more time to detect faults. Due to the low generation rate of transformer fault gases, in order to achieve the equivalent detection performance, the component disclosed by this present application needs more time for fault gas generation compared to conventional detection methods, which affects the real-time performance.

Chinese Patent Application CN104458640A discloses a transformer fault diagnosis method and system based on optical fiber gas online monitoring data. In this patent application, dissolved gases in the oil are filtered into the oil-gas separation device through oil-gas separation material, and then transported to the gas chamber for detection. However, transformer fault gases of extremely low concentrations may not fill up the internal volume of oil-gas separation device and/or gas chamber proposed by this patent application, resulting in lower concentrations detected and inaccurate results.

Japanese Patent Application JPH0552747U discloses a concept of integration of oil-gas separation and detection. However, several issues exist:
1. The patent involves extracting the oil for detection, but cannot detect the gas concentration in real time within the operation condition of the transformer;
2. In this patent, a gap exists between the oil-gas separation membrane and internal gas chamber sensing unit, resulting in unsynchronized diffusion of dissolved gases from the oil-gas separation membrane into the gap, and then from the gap to the internal gas chamber, which may lead to inaccurate detection of the dissolved gas in oil. Additionally, although the gap exists between the oil-gas separation membrane and internal gas chamber sensing unit, due to the low generation rate of fault gases from the oil-immersed transformer, it takes more time to wait for generating sufficient volume of fault gases for detection, causing a certain deviation between the detected and actual fault gas concentrations in oil.
3. The internal gas chamber in this patent uses space to guide light, and the cylindrical unit inside only serves to limit gas diffusion into the air. This increases the energy loss of detection light and reduces the efficiency of signal collection, thereby diminishing detection sensitivity and accuracy.
4. The detection method in this patent is limited to absorption spectroscopy, which analyzes the gas concentration by observing the difference in light intensity before and after absorption. However, when the absorption spectroscopy technology detects the concentration of equivalent nuclear diatoms such as H₂ gas, the absorption signal can be extremely weak, resulting in inaccurate detection.

Therefore, it is of great significance to develop an in-situ detection component of dissolved gas in liquid-phase media (such as transformer oil, gasoline, edible oil, water, etc.), which integrates liquid-gas separation and gas detection (absorption spectroscopy, photoacoustic spectroscopy, photothermal spectroscopy, and Raman spectroscopy), achieving integration and real-time detection.

### SUMMARY

To solve the deficiencies of the existing techniques, the present application provides a solution of an in-situ detection component for dissolved gas in liquid-phase media and the preparation method thereof. By coaxially coating a liquid-gas separation membrane with a hollow-core optical fiber, the present application allows for simultaneous separation and detection of dissolved gases in liquid-phase media, leading to a significant reduction in gas volume required by detection and a substantial improvement in real-time performance, making it possible to achieve accurate in-situ detection of dissolved gas in the early stages of generation of dissolved gases.

The present application adopts the solution as follows:
An in-situ detection component for dissolved gases in liquid-phase media comprises a liquid-gas separation membrane and a drilled hollow-core light guiding device;
The drilled hollow-core light guiding device is tightly and coaxially coated with the liquid-gas separation membrane, realizing simultaneously liquid-gas separation and gas detection;
The dissolved gases in the liquid-phase medium are directly filtered through the liquid-gas separation membrane into the drilled hollow-core light guiding device, which allows laser-gas interactions to generate response signals transmitted through the hollow core, realizing simultaneous dissolved gas separation and detection.

Further, the internal hollow core of the hollow-core light guiding device serving as a chamber for various gas detection techniques such as absorption spectroscopy, photoacoustic spectroscopy, photothermal spectroscopy, and Raman spectroscopy, provides a place for laser and gas interaction and generates gas response signals including absorption signal, photoacoustic signal, photothermal signal and Raman scattering signal respectively, which can reflect the change of gas concentration.

Further, the drilled hollow-core light guiding device has multiple holes perennating through the interior of the hollow-core light guiding device on the coated section coated with the liquid-gas separation membrane;

Where there is no liquid-gas separation membrane coating, no holes are on the surface of the drilled hollow-core light guiding device.

Further, the two ends of the drilled hollow-core light guiding device are respectively aligned and fixed with single-mode solid-core optical fibers through optical fiber sleeves and UV glue.

Further, the ends of the single-mode solid-core optical fibers that are aligned and fixed with the drilled hollow-core light guiding device are coated with visible light high-reflection membrane, and the middle portion of the drilled hollow-core light guiding device coated with the liquid-gas separation membrane forms a resonant cavity of the light guiding device.

Further, the liquid-gas separation membrane is composed of a Teflon AF2400 membrane layer and a mesoporous silica/silane coupling agent layer;

The outer diameter of the drilled hollow-core light guiding device is 300 μm-1 mm, the length is greater than 10 cm, and the ratio of the length to the outer diameter is greater than 333.3.

Further, the mode field diameter of the single-mode solid-core optical fibers aligned and fixed to the two ends of the drilled hollow-core light guiding device is 8-30 μm.

Further, Ta₂O₅/SiO₂ medium coatings serve as the high-reflection membrane with a reflection rate R of >98% in 532-900 nm band.

Further, the distance between the two ends of the drilled hollow-core light guiding device and the single-mode solid-core optical fibers on both sides is 0.3-2 mm;

In order to select the appropriate length L and the reflection rate R of the highly-reflection membrane to obtain the desired signal enhancement factor, according to the parameters above, the theoretical enhancement factor G of the hollow-core light guiding device to signal can be estimated:

$$G = \frac{2 \sqrt{R β}}{1 - R β} \qquad ( 1 )$$

where the R is the highly-reflection membrane's reflection rate, P is the calculated correction factor, which can be expressed as:

$$β = 1 {0}^{- α L / 10} \qquad ( 2 )$$

Where the α is the transmission loss of hollow-core light guiding device, L is the length of the hollow-core light guiding device.

By finely adjusting the distance between the single-mode solid-core optical fibers and the end of the drilled hollow-core light guiding device to ensure that the deviation between the output mode field of the single-mode solid-core optical fibers and the incident mode field of the drilled hollow-core light guiding device does not exceed 10%.

This present application also discloses an in-situ detection device for dissolved gases in liquid-phase media based on the above-mentioned detection component:
The in-situ detection device for dissolved gases in liquid-phase media comprises a laser transmitter and a detector;
The in-situ detection component is placed in the liquid-phase medium containing the dissolved gases to be detected. In the in-situ detection component, the dissolved gases in the liquid-phase medium are filtered into the hollow core region of the drilled hollow-core light guiding device;
The two ends of the in-situ detection component extend out of the container of the liquid-phase medium through optical fibers, with one end connected to the laser transmitter and the other to the detector;
The laser transmitter emits a laser beam that is efficiently coupled into the in-situ detection component, and stimulates gas-laser interaction to generate a gas response signal;
The detector receives and senses the gas response signal within the in-situ detection component.

A method for preparing the above-mentioned in-situ dissolved gas detection component in liquid-phase media is characterized by the following steps:
Step 1, tightly coating a liquid-gas separation membrane on the outer surface of the drilled hollow-core light guiding device;
Step 2, aligning the drilled hollow-core light guiding device coated with the liquid-gas separation membrane and the single-mode solid-core optical fibers through optical fiber sleeves so that the energies can be efficiently coupled;
Step 3, sealing and fixing the whole component using UV glue to obtain in-situ detection component for dissolved gas in the liquid-phase medium.

Compared with existing techniques, the beneficial effects of the present application include the direct, tight coating of the liquid-gas separation membrane on the surface of the drilled hollow-core light guiding device, thus introducing no gap in between, ensuring no additional space for extracted gases to fill up before entering the hollow-core light guiding device of small internal volume and interacting with the laser to generate response signals. The present application realizes a significant reduction in gas volume required by detection and a substantial improvement in real-time performance, making it possible to achieve accurate in-situ detection of dissolved gas in the early stages of gas dissolution.

The present application is not limited to any particular optical detection technique and can be used with a range of techniques, including but not limited to photoacoustic, photothermal, absorption, and Raman spectroscopy, based on actual cases. It ensures fast and accurate in-situ detection for dissolved gas in all liquid-phase media and is useful in various fields, including monitoring the status of energy and power equipment and electrochemical energy storage systems, as well as exploring petroleum and submarine mineral resources.

### BRIEF DESCRIPTION OF THE DRAWINGS

FIG. 1 is the schematic diagram of the in-situ detection component for dissolved gases in liquid-phase media;

FIG. 2 is the schematic diagram of the liquid-gases separation membrane;

FIG. 3 is the flowchart of the preparation process of the in-situ detection component for dissolved gases in liquid-phase media;

FIG. 4 is the flowchart of the detection method for dissolved gases in liquid-phase media using the above-mentioned detection component;

FIG. 5 is the schematic diagram of the detection principle of the in-situ detection component for dissolved gases in liquid-phase media.

### DETAILED DESCRIPTION

The following exemplary embodiments are only intended for a clearer description of the technical solution of the present application and cannot be used to limit the scope of protection.

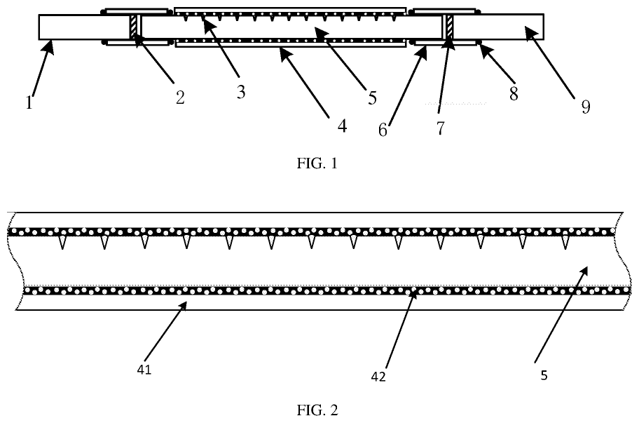

As depicted in FIG. 1, the in-situ detection component for dissolved gases in liquid-phase media is used for integrated liquid-gas separation and accurate concentration detection of dissolved gases. The component comprises a liquid-gas separation membrane 4 and a drilled hollow-core light guiding device 5, which, in combination with single-mode solid-core optical fibers 1 and 9, are firmly sealed by fiber sleeves 6 and UV glue (8) to guarantee the overall sealing of the hollow core light guide device, prevent liquid-phase media from immersing into the hollow-core light guiding device, allowing for fast and accurate in-situ detection of dissolved gases in liquid-phase media.

The hollow-core light guiding device includes, but not limited to, hollow-core optical fiber, hollow-core anti-resonant fiber, photonic bandgap fiber, and internally metal membrane coated capillary.

The drilled hollow-core light guiding device 5 that is tightly and coaxially coated with the liquid-gas separation membrane 4, and the single-mode solid-core optical fibers 1 and the single-mode solid-core optical fibers 9, are aligned by fiber sleeves 6 and fixed by UV glue to form the in-situ detection component for dissolved gases in liquid-phase media.

To further enhance the detection sensitivity of the in-situ detection component for dissolved gases in liquid-phase media, the inner ends of the single-mode solid-core optical fibers 1 and 9 can be coated with visible light high-reflection membrane 2 and visible light high-reflection membrane 7, and together with the drilled hollow-core light guiding device 5, are aligned by fiber sleeves 6 and fixed together by UV glue 8, to form an resonant cavity of light guiding device.

Finely adjusting the distance between the single-mode solid-core optical fibers 1 and 9 and the drilled hollow-core light guiding device 5 to form a stable resonant cavity so that to ensure that the output mode field of the single-mode solid-core optical fiber 1 is approximately consistent with the incident mode field of the drilled hollow-core light guide device 5, and the deviation between the output mode field of the single-mode solid-core optical fiber 1 and the incident mode field of the drilled hollow-core light guiding device 5 does not exceed 10%.

In the exemplary embodiment, the outer diameter of the drilled hollow-core light guiding device 5 is 300 μm-1 mm, transmission lossαis less than 80 dB/km, while the length L is greater than 10 cm, and the ratio of the length to the outer diameter is greater than 333.3. The mode field diameter of the single-mode solid-core optical fibers 1 and 9 is 8-30 μm. Ta₂O₅/SiO₂ medium coatings serve as the high-reflection membrane 2 and 7 with a reflection rate of >98% in 532-900 nm wave band. The distance between the two single-mode solid-core optical fibers 1 and 9 and the two ends of the drilled hollow-core light guiding device 5 is 0.3-2 mm. In order to select the appropriate length L and the reflection rate R of the highly-reflection membrane to obtain a desired signal enhancement factor, according to the parameters above, the theoretical enhancement factor G of the hollow-core light guiding device 5 to the signal can be estimated:

$$G = \frac{2 \sqrt{R β}}{1 - R β} \qquad ( 1 )$$

Where the R is the highly-reflection membrane's reflection rate, β is the calculated correction factor, which can be expressed as:

$$β = 1 {0}^{- α L / 10} \qquad ( 2 )$$

Where the α is the transmission loss of hollow-core light guiding device 5, L is the length of the hollow-core light guiding device 5. The liquid-gas separation membrane 4 tightly adheres to the outer surface of the drilled hollow-core light guiding device 5 by van der Waals forces. When the in-situ detection component is placed in liquid-phase media, the liquid-gas separation membrane 4 directly filters the dissolved gases into the drilled hollow-core light guiding device 5 through holes 3. The response signals produced by the interaction of the laser transmitted in the hollow-core light guiding device 5 and the gases, and the hollow-core light guiding device 5 is used for response signal transmission to realize fast and integrated dissolved gas detection in the liquid-phase media.

The liquid-gas separation membrane 4 also filters impurities in liquid-phase media and enhances the mechanical strength of the drilled hollow-core light guiding device 5.

The response signal is an optical signal that reacts to changes in gases concentration, including absorption signal, photoacoustic signal, photothermal signal, and Raman scattering signal, etc.

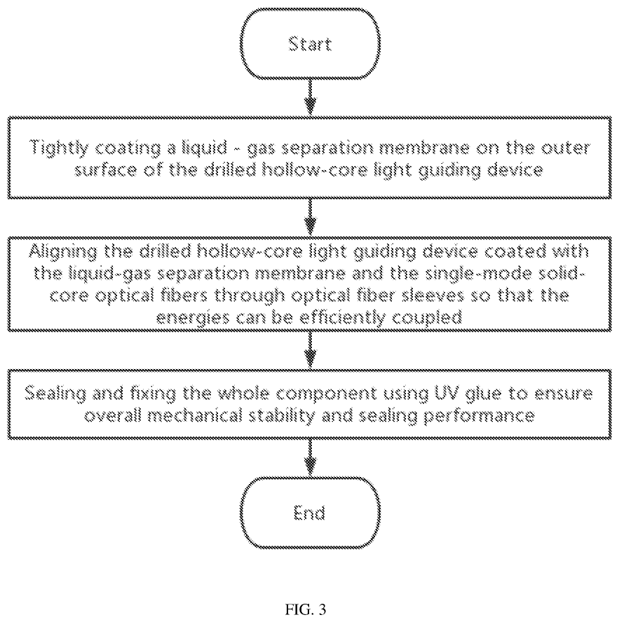

As depicted in FIG. 2, the liquid-gas separation membrane 4 is used to filter dissolved gases in liquid-phase media into the drilled hollow-core light guiding device 5. In one embodiment, the liquid-gas separation membrane 4 comprises a Teflon AF2400 membrane layer 41 and a mesoporous silica/silane coupling agent layer 42 that bonds tightly to the Teflon AF2400 membrane layer 41 with the surface of the drilled hollow-core light guiding device 5 by van der Waals forces.

The liquid-gas separation membrane is not limited to the above-mentioned materials and structure, but includes all materials that have liquid-gas separation function and can tightly adhere to the surface of the hollow-core light guiding device 5.

The selection of the liquid-gas separation membrane material and the optimal thickness are determined by achieving the highest liquid-gas separation efficiency while ensuring mechanical strength. The optimal thickness varies with different liquid-phase media.

The present application also provides an in-situ detection device for dissolved gases in liquid-phase media based on the above-mentioned detection component. The in-situ detection device for dissolved gases in liquid-phase media comprises a laser transmitter and a detector. The in-situ detection component is placed in the liquid-phase medium containing the dissolved gases to be detected. In the in-situ detection component, the dissolved gases in the liquid-phase medium are filtered into the hollow core region of the drilled hollow-core light guiding device 5. The two ends of the in-situ detection component extend out of the container of the liquid-phase medium through optical fibers, with one end connected to the laser transmitter and the other to the detector. The laser transmitter emits a laser beam that is efficiently coupled into the in-situ detection component, and the interaction of the laser and the gas in the hollow core of the in-situ detection component generates a gas response signal. The detector receives and senses the gas response signal within the in-situ detection component.

As depicted in FIG. 3, the present application also provides a method for preparing the in-situ detection component for dissolved gases in liquid-phase media, including the following steps:

(1) tightly coating a liquid-gas separation membrane 4 on the outer surface of the drilled hollow-core light guiding device 5.

As an example, the hollow-core anti-resonant fiber is used as the hollow-core light guiding device 5, with a diameter of 28 μm of the hollow-core fiber and a length of greater than 10 cm, exhibiting a loss of 80 dB/km at the wavelength of 532 nm.

(2) aligning the drilled hollow-core light guiding device 5 coated with the liquid-gas separation membrane 4 and the single-mode solid-core optical fibers through optical fiber sleeves 6 so that the energies can be efficiently coupled, with a distance of approximate 0.1 mm in between.

The single-mode solid-core optical fibers 1 and 9 can be SMF-28 single-mode solid-core optical fibers which is commonly used in engineering practice, with a mode field diameter of 10 μm.

Customizing the size of fiber sleeve 6, including optimizing the hole size at both ends of the fiber sleeve 6 to ensure the coaxial positioning of the hollow-core light guiding device 5 and the single-mode solid-core optical fibers coated with high-reflection membrane.

(3) sealing and fixing the whole component with UV glue 8 to form a precision drilled hollow-core light guiding device resonant cavity to prevent liquid from entering the hollow-core light guiding resonant cavity and ensure the overall mechanical stability and sealing performance.

The present application also provides a method for constructing an enhanced in-situ detection component for dissolved gases in liquid-phase media, including the following steps:
(1) coating high-reflection membrane 2 and 7 on the ends of single-mode solid-core optical fibers 1 and 9 respectively as the mirrors of resonant cavity;
Ta2O5/SiO2 medium coatings serve as the high-reflection membrane 2 and 7 with a reflection rate of >98% in 532-900 nm wave band;
In the embodiment, 13 layers of Ta2O5/SiO2 medium coatings are applied.
(2) the two single-mode solid-core optical fibers coated with high-reflection membrane on inner ends, and together with the drilled hollow-core light guiding device coated with liquid-gas separation membrane on the middle portion, are aligned by fiber sleeves, fixed together by UV glue, and frequency-locked to form a drilled hollow-core light guiding device resonant cavity.

The present application also provides a method for preparing of the liquid-gas separation membrane including the following steps:
(1) mixing mesoporous silica and silane coupling agent with a certain mass ratio to obtain a mesoporous silica/silane coupling agent mixture solution;
Mesoporous silica is a solid, and silane coupling agent is a commercially available standard solution. The two are mixed at different mass ratios;
The mass ratio of mesoporous silica to silane coupling agent is modified as needed to obtain in-situ detection components corresponding to different mass ratios, and the in-situ detection components is used for liquid-gas separation of the measured liquid medium. The best mass ratio is determined by analyzing the relationship between the mass ratio and permeability.
(2) coating the surface of the drilled hollow-core light guiding device with the mesoporous silica/silane coupling agent mixture solution, followed by putting it into a drying oven for drying procedure with horizontal rotation to form the mesoporous silica/silane coupling agent layer 42. The layer thickness of mesoporous silica/silane coupling agent lay 42 can be controlled by controlling the number of coatings;
The best mass ratio and thickness of the mesoporous silica/silane coupling agent layer 42 for optimal permeability and bonding strength are determined by analyzing the permeability and bonding strength of the different mass ratios and thicknesses of the mesoporous silica/silane coupling agent layer 42 to the surface of the hollow-core light guiding device 5;
The silane coupling agent has two groups of affinities for inorganic materials and organic materials respectively, which can ensure tight adhesion between the Teflon AF2400 membrane layer and the drilled hollow-core light guiding device by van der Waals forces; the mesoporous silica is used to increase the specific surface area of the membrane material and improve the permeability of the membrane material.
(3) coating the mesoporous silica/silane coupling agent layer 42 with a 1% solution by weight of Teflon AF2400, followed by putting it into the drying oven for drying procedure with horizontal rotation to form Teflon AF2400 membrane layer 41. The layer thickness can be controlled by controlling the number of coatings;
(4) placing the drilled hollow-core light guiding device 5 with the liquid-gas separation membrane 4 in the liquid-phase medium to be detected and analyze the effect of different Teflon AF2400 membrane layer thicknesses on the liquid-gas separation time to determine the thickness of the Teflon AF2400 membrane layer 41.

The optimal thickness of the Teflon AF2400 membrane layer 41, which varies with different liquid-phase media, is based on achieving the highest liquid-gas separation efficiency while ensuring mechanical strength.

The optimal thickness of the mesoporous silica/silane coupling agent layer 42 is based on minimizing the time for dissolved gas to enter the drilled hollow-core light guiding device 5 while ensuring tight adhesion between the Teflon AF2400 membrane 41 and the drilled hollow-core light guiding device 5.

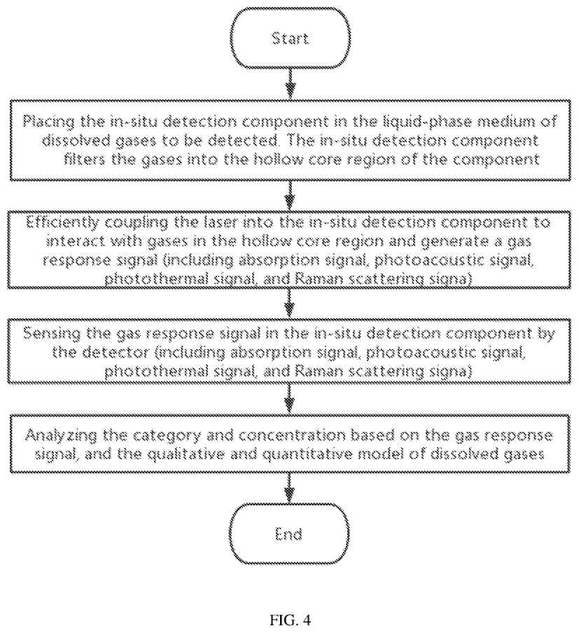

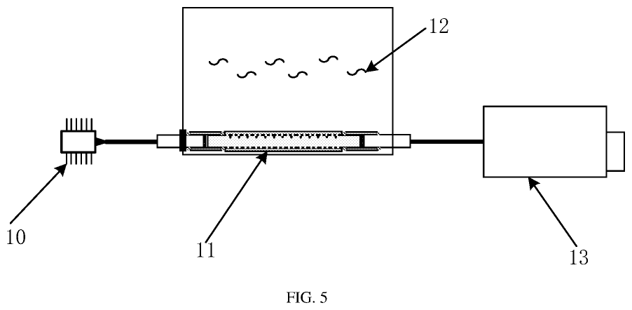

As depicted in FIG. 4, the in-situ detection method for dissolved gas based on the detection component of the present application includes the following steps:
(1) placing the in-situ detection component 11 in the liquid phase medium 12 containing the dissolved gases to be detected. The in-situ detection component filters the gases into the hollow core region of the component 11. The detection principle of the in-situ detection component is as depicted in FIG. 5;
(2) efficiently coupling the laser beam emitted by the laser transmitter 10 into the in-situ detection component 11, interacting with the gases in the hollow core of the in-situ detection component, and generating a gas response signal, including absorption signal, photoacoustic signal, photothermal signal, and Raman scattering signal, etc., which can reflect the concentration of the gases;
(3) the detector 13 sensing the gas response signal in the in-situ detection component 11, including absorption signal, photoacoustic signal, photothermal signal, Raman signal, etc.;
(4) analyzing the category and concentration of the dissolved gas based on the gas response signal, and the qualitative and quantitative model of dissolved gases, thereby achieving in-situ dissolved gas detection.

Compared with existing techniques, the advantages of the present application include the direct, tight coating of the liquid-gas separation membrane on the surface of the drilled hollow-core light guiding device, thus introducing no gap in between, ensuring no additional space for extracted gases to fill up before entering the hollow-core light guiding device of small internal volume and interacting with the laser to generate response signals. The present application realizes a significant reduction in gas volume required by detection and a substantial improvement in real-time performance, making it possible to achieve accurate in-situ detection of dissolved gas in the early stages of generation.

The present application is not limited to any particular optical detection technique and can be used with a range of techniques, including but not limited to photoacoustic, photothermal, absorption, and Raman spectroscopy, based on actual cases. It ensures fast and accurate in-situ dissolved gas detection in all liquid-phase media and is useful in various fields, including monitoring the status of energy and power equipment and electrochemical energy storage systems, as well as exploring petroleum and submarine mineral resources.

The applicant of the present application has provided a detailed explanation and description of the embodiments of the present application in conjunction with the figures in the specification. However, those skilled in the art should understand that the above-mentioned embodiments are only preferred embodiments of the present application. The detailed description is only to help readers better understand the spirit of the present application and is not a limitation on the scope of the present application. On the contrary, any improvements or modifications based on the spirit of the present application should fall within the scope of the present application's protection.

1. An in-situ detection component for dissolved gases in liquid-phase medium, comprising a liquid-gas separation membrane and a drilled hollow-core light guiding device, wherein:
the drilled hollow-core light guiding device tightly and coaxially coating with the liquid-gas separation membrane to realize simultaneous dissolved gases separation and detection;
the liquid-gas separation membrane filtering the dissolved gases in the liquid-phase medium into the drilled hollow-core light guiding device directly;
the drilled hollow-core light guiding device transmitting a laser and a gas response signal, which realizes simultaneous dissolved gases separation and detection.

2. The in-situ detection component for the dissolved gases in the liquid-phase medium of claim 1, wherein:
an internal hollow core of the hollow-core light guiding device serves as a chamber for various detection techniques such as absorption spectroscopy, photoacoustic spectroscopy, photothermal spectroscopy, and Raman spectroscopy, provides a space for laser and gases interaction and generates gas response signals, which reflects the change of gas concentration, including absorption signal, photoacoustic signal, photothermal signal and Raman scattering signal.

3. The in-situ detection component for the dissolved gases in the liquid-phase medium of claim 1, wherein:
the drilled hollow-core light guiding device has multiple holes penetrating through the interior of the hollow-core light guiding device on a coated section with the liquid-gas separation membrane;
no holes positioning on surface of two ends of the drilled hollow-core light guiding device where no liquid-gas separation membrane coating.

4. The in-situ detection component for the dissolved gases in the liquid-phase medium of claim 3, wherein:
the two ends of the drilled hollow-core light guiding device are aligned and fixed with single-mode solid-core optical fibers through optical fiber sleeves and UV glue respectively.

5. The in-situ detection component for the dissolved gases in the liquid-phase medium of claim 4, wherein:
the ends of the single-mode solid-core optical fibers that are aligned and fixed with the drilled hollow-core light guiding device are coated with visible light high-reflection membrane;
the drilled hollow-core light guiding device coated with the liquid-gas separation membrane forms a light guiding device resonant cavity.

6. The in-situ detection component for the dissolved gases in the liquid-phase medium of claim 1, wherein:
the liquid-gas separation membrane is composed of a Teflon AF2400 membrane layer and a mesoporous silica/silane coupling agent layer.

7. The in-situ detection component for the dissolved gases in the liquid-phase media of claim 6, wherein:
the outer diameter of the drilled hollow-core light guiding device is 300 μm to 1 mm, the length of the drilled hollow-core light guiding device is greater than 10 cm, and the ratio of the length to the outer diameter is greater than 333.3.

8. The in-situ detection component for the dissolved gases in the liquid-phase medium of claim 7, wherein:
the mode field diameter of the single-mode solid-core optical fibers aligned and fixed to the two ends of the drilled hollow-core light guiding device is 8 to 30 m.

9. The in-situ detection component for the dissolved gases in the liquid-phase medium of claim 8, wherein:
the high-reflection membrane is Ta₂O₅/SiO₂ medium coatings, which has a reflection rate higher than 98% in 532 to 900 nm wave band.

10. The in-situ detection component for the dissolved gases in the liquid-phase medium of claim 9, wherein:
the distance between the two single-mode solid-core optical fibers and the two ends of the drilled hollow-core light guiding device is 0.3 to 2 mm;
by finely adjusting the distance between the single-mode solid-core optical fibers and the ends of the drilled hollow-core light guiding device to ensure that the deviation between the output mode field of the single-mode solid-core optical fibers and the incident mode field of the drilled hollow-core light guiding device is not more than 10%.

11. An in-situ detection device for dissolved gases in liquid-phase medium, comprising a laser transmitter and a detector;
when placing the in-situ detection component in the liquid-phase medium containing the dissolved gases to be detected; the dissolved gases in the liquid-phase medium are filtered into a hollow core region of the drilled hollow-core light guiding device in the in-situ detection component;
two ends of the in-situ detection component extend out of a container of the liquid-phase medium through optical fibers, with one end connected to the laser transmitter and the other end connected to the detector;
the laser transmitter emits a laser beam that is efficiently coupled into the in-situ detection component, and the laser beam and the gas in the hollow core of the in-situ detection component interact to generate a gas response signal;
the detector receives and senses the gas response signal within the in-situ detection component.

12. A method for preparing the in-situ detection component for dissolved gases in liquid-phase medium, comprising:
Step 1, tightly coating a liquid-gas separation membrane on an outer surface of a drilled hollow-core light guiding device;
Step 2, aligning the drilled hollow-core light guiding device coated with the liquid-gas separation membrane and single-mode solid-core optical fibers through optical fiber sleeves so that energies can be efficiently coupled;
Step 3, sealing and fixing the whole component using UV glue to obtain in-situ detection component for dissolved gas in the liquid-phase medium.
